(19) United States
(12) Patent Application Publication
(10) Pub. No.: US 2010/0160401 A1
(43) Pub. Date: Jun. 24, 2010

(54) 4-PHENYLIMIDAZOLE-2-THIONE TYROSINASE INHIBITORS AND TREATMENT OR PREVENTION OF PIGMENTARY DISORDERS THEREWITH

(75) Inventors: Jean-Guy BOITEAU, Valbonne (FR); Isabelle Pelisson, Vallauris (FR); Itaru Suzuki, Fayence (FR); Branislav Musicki, Nice (FR); Marielle Berthier, Nice (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT, FRANCE

(21) Appl. No.: 12/631,450

(22) Filed: Dec. 4, 2009

Related U.S. Application Data
(63) Continuation of application No. PCT/FR2008/050993, filed on Jun. 4, 2008.

(30) Foreign Application Priority Data
Jun. 5, 2007 (FR) 0755468
Jun. 4, 2008 (FR) PCT/FR2008/050993

(51) Int. Cl. A61K 31/415

(52) U.S. Cl. 514/392

(57) ABSTRACT

The tyrosinase inhibiting compounds of the following general formula

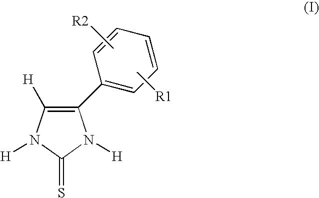

formulated into pharmaceutical or cosmetic compositions, are useful for the treatment or prevention of pigmentary disorders, or for preventing and/or treating signs of skin aging, and/or for body or hair hygiene.

### CROSS-REFERENCE TO PRIORITY/PCT APPLICATIONS

This application claims priority under 35 U.S.C. §119 of FR 08/55205, filed Nov. 30, 2008, and is a continuation/national phase of PCT/FR 2009/052432, filed Nov. 30, 2009 and designating the United States (published in the French language on Jun. 5, 2009 as WO 2009/065316 A2; the title and abstract were also published in English), each hereby expressly incorporated by reference in its entirety and each assigned to the assignee hereof.

### BACKGROUND OF THE INVENTION

1. Technical Field of the Invention

The invention relates to the administration of 4-phenylimidazole-2-thione compounds as tyrosinase inhibitors, formulated into pharmaceutical or cosmetic compositions, for the treatment or prevention of pigmentary disorders.

2. Description of Background and/or Related and/or Prior Art

The pigmentation of the skin, in particular human skin, results from the synthesis of melanin by the dendritic cells, the melanocytes. The melanocytes comprise organelles, known as melanosomes, which transfer the melanin into the upper layers of keratinocytes, which are then transported to the surface of the skin via the differentiation of the epidermis (Gilchrest B A, Park H Y, Eller M S, Yaar M, Mechanisms of ultraviolet light-induced pigmentation. Photochem Photobiol., 1996; 63: 1-10; Hearing V J, Tsukamoto K, Enzymatic control of pigmentation in mammals. FASEB J., 1991; 5: 2902-2909).

Among the enzymes of melanogenesis, tyrosinase is a key enzyme which catalyzes the first two stages of the synthesis of melanin. Homozygous tyrosinase mutations result in oculocutaneous albinism type I characterized by a complete absence of the synthesis of melanin (Toyofuku K, Wada I, Spritz R A, Hearing V J, The molecular basis of oculocutaneous albinism type 1 (OCA1): sorting failure and degradation of mutant tyrosinases results in a lack of pigmentation. Biochem J 2001; 355: 259-269).

It is proving to be important to develop novel therapeutic approaches to treat disorders of pigmentation resulting from an increase in the production of melanin, for which there exists no treatment satisfying all the expectations of patients and dermatologists.

The majority of skin-lightening compounds already known are phenols/catechols. These compounds inhibit tyrosinase but the majority of them are cytotoxic to the melanocytes owing to the formation of quinones. This toxic effect risks bringing about permanent depigmentation of the skin.

### SUMMARY OF THE INVENTION

It has now unexpectedly and surprisingly been discovered that certain 4-phenylimidazole-2-thiones already known to the prior art exhibit a very good inhibitory activity for the enzyme tyrosinase and a very low cytotoxicity.

These compounds find applications in human medicine, in particular in dermatology, and in the field of cosmetics.

Among the imidazole-2-thione derivatives already known, some have been described as having anti-inflammatory properties (S. Maeda, M. Suzuki, T. Iwasaki, K. Matsumoto and Y. Iwazawa, Chem. Pharm. Bull., 1984, 32, 7, 2536-2543).

Others have been reported, in U.S. Pat. No. 4,798,843, to be dopamine-beta-hydroxylase inhibitors. The compounds described in this patent are effective in preventing gastric ulcers.

Other imidazole-2-thiones are also described as reaction intermediates in the synthesis of H3 receptor antagonists (M. Mor, F. Bordi, C. Silva, S. Rivara, P. Crivori, P. V. Plazzi, V. Ballabeni, A. Caretta, E. Barocelli, M. Impicciatore, P.-A Carrupt, and B. Testa J. Med. Chem. 1997, 40, 2471-2578).

Others have been described, in WO 2006/019962, as modulators of TLR and TNF-alpha. In this patent publication, the compounds claimed are useful in the treatment of IBD (inflammatory bowel disease) and gastrointestinal pathological conditions.

EP131973 also teaches the administration of certain compounds derived from imidazole-2-thiones as inhibitors of gastric acid secretion that are useful in the treatment against ulcers.

JP05132422 discloses the administration of certain imidazole-2-thiones as tyrosinase inhibitors. However, no imidazole-2-thione derivative substituted in the 4 position by an aryl moiety is described. No inhibitory activity for tyrosinase is shown for compounds with the 4-arylimidazole-2-thione structure. In point of fact, it has now unexpectedly and surprisingly been found that certain compounds with the 4-phenylimidazole-2-thione structure according to the present invention exhibit an inhibitory activity for tyrosinase which is much better than that of the compounds of JP05132422.

Thus, the present invention features administration of the compounds of the following general formula (I), formulated into pharmaceutical compositions for the treatment or prevention of pigmentary disorders:

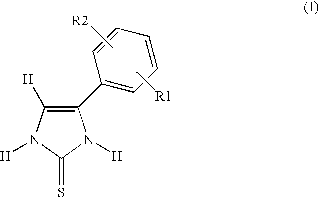

wherein:

R1 and R2, which may be identical or different, are each:

a hydrogen atom,

a C₁-C₇ alkyl radical,

a hydroxymethyl, trifluoromethoxy, difluoromethoxy or trifluoromethyl radical,

a C₃-C₇ cycloalkyl radical, with the proviso that one of the carbon atoms of the ring may optionally be replaced by an oxygen or sulfur atom,

a C₄-C₉ cycloalkylalkyl radical,

a morpholinyl, thiomorpholinyl, piperazinyl or N-methylpiperazinyl group,

a carboxyl substituent,

a (C₁-C₄ alkoxy)carbonyl group,

a C₁-C₆ alkoxy radical, or

a halogen atom,

with the proviso that, when R2 is in the ortho position with respect to R1, then R1 and R2, which may be identical or different, are each a C₁-C₅ alkyl radical, or form a hydrocarbon ring containing 5 or 6 atoms, and also with the proviso that 1 or 2 carbon atom(s) of said hydrocarbon ring can optionally be replaced by 1 or 2 oxygen atom(s), and the salts of the compounds of formula (I) and the tautomeric forms thereof.

### DETAILED DESCRIPTION OF BEST MODE AND SPECIFIC/PREFERRED EMBODIMENTS OF THE INVENTION

The tautomeric forms can be represented as follows:

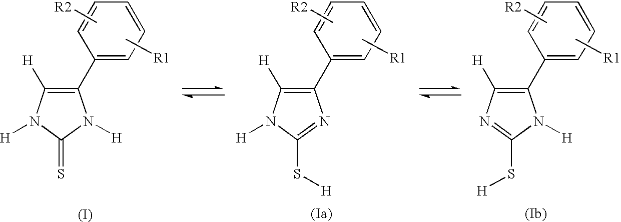

Preferred are, among the addition salts of the compounds of general formula (I) with a pharmaceutically acceptable acid, the salts with an organic acid or with an inorganic acid.

The appropriate inorganic acids are, for example, hydrohalic acids, such as hydrochloric acid or hydrobromic acid, sulfuric acid or nitric acid.

The appropriate organic acids are, for example, picric acid, methanesulfonic acid, ethanesulfonic acid or trifluoromethanesulfonic acid.

The compounds of general formula (I) can also exist in the form of hydrates or of solvates with water or with a solvent.

The appropriate solvents for forming solvates or hydrates are, for example, alcohols, such as ethanol or isopropanol, or water.

According to the present invention, C₃-C₇ cycloalkyl is a saturated cyclic hydrocarbon chain having from 3 to 7 carbon atoms. Preferably, the C₃-C₇ cycloalkyl radical is selected from among cyclopropyl, cyclobutyl, cyclopentyl, cyclohexyl and cycloheptyl radicals.

According to the present invention, C₁-C₇ alkyl is a saturated and linear or branched hydrocarbon chain having from 1 to 7 carbon atoms. Preferably, the C₁-C₇ alkyl radical is selected from among methyl, ethyl, propyl, isopropyl, butyl, t-butyl, pentyl, hexyl and heptyl radicals.

According to the present invention, C₄-C₉ cycloalkylalkyl is a saturated and linear or branched hydrocarbon chain substituted by a cycloalkyl radical and having from 4 to 9 carbon atoms. Preferably, the C₄-C₉ cycloalkylalkyl radical is selected from among cyclopropylmethyl, cyclopropylethyl, cyclobutylmethyl, cyclobutylethyl, cyclopentylmethyl, cyclopentylethyl, cyclohexylmethyl and cyclohexylethyl radicals.

According to the present invention, (C₁-C₄ alkoxy)carbonyl is a carboxyl radical substituted by an alkyl radical having from 1 to 4 carbon atoms. Preferably, the (C₁-C₄ alkoxy)carbonyl radical is selected from among methoxycarbonyl, ethoxycarbonyl, propoxycarbonyl and butoxycarbonyl radicals.

According to the present invention, C₁-C₆ alkoxy is an oxygen atom substituted by a saturated and linear or branched hydrocarbon chain having from 1 to 6 carbon atoms. Preferably, the C₁-C₆ alkoxy radical is selected from among methoxy, ethoxy, propoxy, butoxy, pentoxy and hexyloxy radicals.

According to the present invention, halogen is a fluorine, chlorine or bromine atom.

According to the present invention, the compounds of general formula (I) that are preferred are those for which R1 and R2, which may be identical or different, are each a hydrogen atom or a C₁-C₇ alkyl radical or a C₃-C₇ cycloalkyl radical.

According to the present invention, the compounds of general formula (I) which are particularly preferred are those for which:

R2 is a hydrogen atom, and

R1 is a hydrogen atom or a C₁-C₇ alkyl radical or a C₃-C₇ cycloalkyl radical.

Among the compounds of formula (I) according to the present invention, the following compounds are particularly exemplary:
1. 4-phenyl-1,3-dihydroimidazole-2-thione: RN6857-34-7;
2. 4-phenyl-1,3-dihydroimidazole-2-thione hydrochloride: RN93168-73-1;
3. 4-(4-methoxyphenyl)-1,3-dihydroimidazole-2-thione: RN10486-41-6;
4. 4-(3-bromophenyl)-1,3-dihydroimidazole-2-thione: RN192800-59-2;
5. 4-(4-bromophenyl)-1,3-dihydroimidazole-2-thione: RN 436095-86-2;
6. 4-(2-fluorophenyl)-1,3-dihydroimidazole-2-thione: RN93103-13-0;
7. 4-(3-fluorophenyl)-1,3-dihydroimidazole-2-thione: RN93103-14-1;
8. 4-(4-fluorophenyl)-1,3-dihydroimidazole-2-thione: RN93103-15-2;
9. 4-(2-chlorophenyl)-1,3-dihydroimidazole-2-thione: RN93103-16-3;
10. 4-(3-chlorophenyl)-1,3-dihydroimidazole-2-thione: RN93103-17-4;
11. 4-(4-chlorophenyl)-1,3-dihydroimidazole-2-thione: RN93103-18-5;
12. 4-(4-butoxyphenyl)-1,3-dihydroimidazole-2-thione: RN192800-50-3;
13. 4-(4-methylphenyl)-1,3-dihydroimidazole-2-thione: RN93103-19-6;
14. 4-(4-trifluoromethylphenyl)-1,3-dihydroimidazole-2-thione: RN38575-47-2;
15. 4-(4-isopropylphenyl)-1,3-dihydroimidazole-2-thione: RN93103-20-9;
16. 4-(4-propylphenyl)-1,3-dihydroimidazole-2-thione: RN192800-52-5;
17. 4-(4-tert-butylphenyl)-1,3-dihydroimidazole-2-thione: RN93103-21-0;
18. 4-(3,4-dimethoxyphenyl)-1,3-dihydroimidazole-2-thione: RN117877-37-9; and
19. 4-(3-propyloxyphenyl)-1,3-dihydroimidazole-2-thione: RN192800-64-9.

Advantageously, the compounds of the present invention exhibit an IC₅₀ (dose which inhibits 50% of the enzymatic activity) value with regard to tyrosinase of less than or equal to 10 μM and more particularly of less than or equal to 1 μM.

This invention therefore features formulation of at least one compound of general formula (I) into pharmaceutical or cosmetic compositions exhibiting tyrosinase-inhibiting activity.

This invention also features administration of the compounds of formula (I) for the treatment and/or prevention of pigmentary disorders.

The present invention also features a therapeutic or cosmetic treatment regime or regimen comprising the administration, as tyrosinase inhibitor, of a pharmaceutical or cosmetic composition comprising the said compounds for formula (I).

The present invention also features the formulation of a compound of general formula (I) into medicaments useful in the treatment of pigmentary disorders, and preferably of hyperpigmentary disorders.

This because the compounds according to the invention are particularly appropriate for the treatment and/or prevention of pigmentary disorders, and preferably of hyperpigmentary disorders, such as melasma, chloasma, lentigines, senile lentigo, irregular hyperpigmentations related to photoageing, freckles, postinflammatory hyperpigmentations due to an abrasion and/or a burn and/or a scar and/or a dermatosis and/or a contact allergy; naevi, genetically determined hyperpigmentations, hyperpigmentations of metabolic or drug origin, melanomas or any other hyperpigmentary lesion.

Another aspect of the present invention is a pharmaceutical composition for use in particular in the treatment of the above-mentioned conditions and which comprises, formulated into a pharmaceutically acceptable vehicle compatible with the method of administration selected for the composition, at least one compound of general formula (I) in one of its tautomeric forms or at least one of its salts with a pharmaceutically acceptable acid.

“Pharmaceutically acceptable vehicle” means a medium compatible with the skin, mucous membranes and superficial body growths.

The compositions according to the invention can be administered topically. Preferably, the pharmaceutical composition is packaged in a form suitable for application topically. Topically means administration to the skin or mucous membranes.

The pharmaceutical compositions according to the invention are administered topically, more particularly for the treatment of the skin and mucous membranes and can be provided in the liquid, pasty or solid form and more particularly in the form of ointments, creams, milks, salves, powders, impregnated pads, syndets, solutions, gels, sprays, foams, suspensions, sticks, shampoos or washing bases. Same can also be provided in the form of suspensions of microspheres or nanospheres or of lipid or polymeric vesicles or of polymeric or gelled patches which make possible controlled release.

The compositions for topical application have a concentration of compound according to the invention generally ranging from 0.001% to 10% by weight, preferably from 0.01% to 5% by weight, with respect to the total weight of the composition.

The compounds of general formula (I) according to the invention also find application in the cosmetics field, in particular in protecting against the harmful aspects of the sun, for preventing and/or combating photoinduced or chronological aging of the skin and superficial body growths.

Another aspect of the invention is thus compositions comprising, in a cosmetically acceptable vehicle, at least one of the compounds of general formula (I). “Cosmetically acceptable vehicle” means a medium compatible with the skin, mucous membranes and superficial body growths.

Another aspect of the invention is the cosmetic application of a compound of formula (I) or of a composition comprising at least one compound of general formula (I) for preventing and/or treating signs of skin aging.

Another aspect of the invention is the cosmetic application of a compound of formula (I) or of a composition comprising at least one compound of general formula (I) for body or hair hygiene.

The cosmetic compositions according to the invention comprising, in a cosmetically acceptable vehicle, a compound of general formula (I) or one of its tautomeric forms or one of its salts with a pharmaceutically acceptable acid can be provided in particular in the form of a cream, a milk, a gel, suspensions of microspheres or nanospheres or lipid or polymeric vesicles, impregnated pads, solutions, sprays, foams, sticks, soaps, washing bases or shampoos.

The concentration of compound of general formula (I) in the cosmetic composition preferably ranges from 0.001% to 10% by weight, with respect to the total weight of the composition.

The pharmaceutical and cosmetic compositions as described above can additionally comprise inert additives or even pharmacodynamically active additives, as regards the pharmaceutical compositions, or combinations of these additives, and in particular:

wetting agents;

flavor enhancers;

preservatives, such as para-hydroxybenzoic acid esters;

stabilizers;

moisture regulators;

pH-regulating agents;

osmotic pressure modifiers;

emulsifying agents;

UV-A and UV-B screening agents;

antioxidants, such as α-tocopherol, butylated hydroxyanisole or butylated hydroxytoluene, superoxide dismutase or ubiquinol;

emollients;

moisturizing agents, such as glycerol, PEG 400, thiamorpholinone and its derivatives, or urea;

anti-seborrhoeic or anti-acne agents, such as S-carboxymethylcysteine, S-benzylcysteamine, their salts or their derivatives, or benzoyl peroxide.

Of course, one skilled in the art will take care to select the optional compound or compounds to be added to these compositions such that the advantageous properties intrinsically associated with the present invention are not, or not substantially, detrimentally affected by the envisaged addition.

To further illustrate the present invention and the advantages thereof, the following specific examples are given, including those indicating biological activity, it being understood that same are intended only as illustrative and in nowise limitative. In said examples to follow, all parts and percentages are given by weight, unless otherwise indicated.

### Example 1

### Tyrosinase Activity Inhibition Assay

The activity of the inhibitors is measured starting from a lysate of B16F1 cells (murine melanoma line). In the presence of the L-tyrosine substrate, the tyrosinase present in these cells catalyses the hydroxylation of L-tyrosine to give L-DOPA and then the oxidation of the L-DOPA to give dopaquinone. In the presence of MBTH (3-methyl-2-benzothiazolinone hydrazone), the dopaquinone is trapped so as to form a pink complex which absorbs at 520 nm.

The B16F1 cells are cultured in DMEM medium+10% foetal calf serum+10⁻⁹ M α-MSH for 4 days at 37° C. under 7% CO₂. They are treated with trypsin, washed with PBS, counted and pelleted. The pellet is taken up at 10⁷ cells/ml in lysis buffer (10 mM sodium phosphate, pH 6.8-1% Igepal) and the suspension is treated with ultrasound for 10 seconds. After centrifugation for 30 minutes at 4000 rpm, the supernatant obtained constitutes the cell lysate used as tyrosinase source in the enzymatic assay.

The assays are carried out in duplicate in 384-well plates in a total volume of 50 μl. Each well contains:

40 μl of solution containing 1.25 mM L-tyrosine, 6.25 μM L-DOPA (cofactor) and 3.75 mM MBTH in buffer B (62.25 mM sodium phosphate, pH 6.8-2.5% dimethylformamide),

5 μl of inhibitor diluted in DMSO,

5 μl of cell lysate diluted to ½ in 50 mM Tris HCl buffer, pH 7.5.

The plate is incubated at 37° C. and a spectrophotometric reading is carried out at 520 nm after incubating for 6 hours. To avoid any possible absorption of the products, the system uses corrected absorbance (absorbance at time 6 h absorbance at time zero).

The inhibitors are assayed in terms of dose-response so as to calculate an IC₅₀ (dose which inhibits 50% of the enzymatic activity).

Several internal controls are added to each experiment:

control for 100% activity: the 5 μl of inhibitor are replaced with 5 μl of DMSO,

control for 50% activity: the 5 μl of inhibitor are replaced with 5 μl of phenylthiourea at 300 μM in DMSO,

control for 0% activity: the L-tyrosine substrate is replaced with buffer B.

The results obtained for the compounds of the invention are presented in Table A:

TABLE A

| | | Tyrosine |
|---|---|---|
| | | hydroxylase/Dopa |
| Name | Structure | oxidase IC₅₀ (μM) |
| Compound 1 | 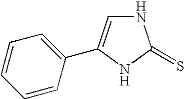 | 0.25 |
| Compound 3 | 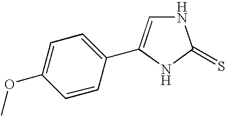 | 0.9 |
| Compound 4 | 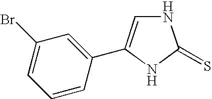 | 3 |
| Compound 8 | 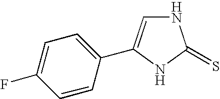 | 0.3 |
| Compound 13 | 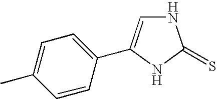 | 0.6 |

### Example 2

### Formulations

In this example, various specific formulations based on the compounds according to the invention have been illustrated.

Topically:

(a) Ointment:

| | |
|---|---|
| Compound 1 | 10.020 g |
| Isopropyl myristate | 81.700 g |
| Liquid petrolatum | 9.100 g |
| Silica (“Aerosil 200”) | 9.180 g |

(b) Ointment:

| | | |
|---|---|---|
| Compound 6 | 60.300 | g |
| White petrolatum, pharmaceutical grade | q.s. for 100 | g |

(c) Nonionic Water-In-Oil Cream:

| | | |
|---|---|---|
| Compound 1 | 10.100 | g |
| Mixture of emulsive lanolin alcohols, | 39.900 | g |
| of waxes and of oils (“Anhydrous eucerin”) | | |
| Methyl para-hydroxybenzoate | 0.075 | g |
| Propyl para-hydroxybenzoate | 0.075 | g |
| Sterile demineralized water | q.s. for 100 | g |

(d) Lotion:

| | |
|---|---|
| Compound 6 | 60.100 g |
| Polyethylene glycol (PEG 400) | 69.900 g |
| 95% Ethanol | 30.000 g |

(e) Hydrophobic Ointment:

| | | |
|---|---|---|
| Compound 2 | 20.300 | g |
| Isopropyl myristate | 36.400 | g |
| Silicone oil (“Rhodorsil 47 V 300”) | 36.400 | g |
| Beeswax | 13.600 | g |
| Silicone oil (“Abil 300 000 cst”) | q.s. for 100 | g |

(f) Nonionic Oil-In-Water Cream:

| | | |
|---|---|---|
| Compound 4 | 41.000 | g |
| Cetyl alcohol | 4.000 | g |
| Glycerol monostearate | 2.500 | g |
| PEG 50 stearate | 2.500 | g |
| Shea butter | 9.200 | g |
| Propylene glycol | 2.000 | g |
| Methyl para-hydroxybenzoate | 0.075 | g |
| Propyl para-hydroxybenzoate | 0.075 | g |
| Sterile demineralized water | q.s. for 100 | g |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for the treatment and/or prevention of pigmentary disorders, comprising topically applying onto the skin, mucous membranes and/or superficial body growth of an individual in need of such treatment, a thus effective amount of at least one tyrosinase inhibiting compound of general formula (I): 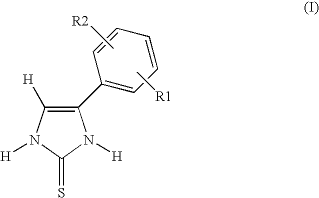
wherein:
R1 and R2, which may be identical or different, are each:
a hydrogen atom,
a C₁-C₇ alkyl radical,
a hydroxymethyl, trifluoromethoxy, difluoromethoxy or trifluoromethyl radical,
a C₃-C₇ cycloalkyl radical, with the proviso that one of the carbon atoms of the ring may be optionally replaced by an oxygen or sulfur atom,
a C₄-C₉ cycloalkylalkyl radical,
a morpholinyl, thiomorpholinyl, piperazinyl or N-methylpiperazinyl group,
a carboxyl substituent,
a (C₁-C₄ alkoxy)carbonyl group,
a C₁-C₆ alkoxy radical, or
a halogen atom,
with the proviso that, when R2 is in the ortho position with respect to R1, then R1 and R2, which may be identical or different, are each a C₁-C₅ alkyl radical, or form a hydrocarbon ring containing 5 or 6 atoms, and also with the proviso that 1 or 2 carbon atom(s) of said hydrocarbon ring can optionally be replaced by 1 or 2 oxygen atom(s),
and/or the salts of the compounds of formula (I) and/or the tautomeric forms thereof, formulated into a topically applicable, pharmaceutically/cosmetically acceptable vehicle therefor.

2. The regime or regimen as defined by claim 1, wherein the at least one compound of formula (I) comprises a salt formed with a pharmaceutically acceptable acid, said acid being selected from among inorganic acids and organic acids.

3. The regime or regimen as defined by claim 1, wherein the at least one compound of formula (I) comprises a hydrate or a solvate.

4. The regime or regimen as defined by claim 1, said at least one compound of formula (I) comprising a C₃-C₇ cycloalkyl radical selected from among cyclopropyl, cyclobutyl, cyclopentyl, cyclohexyl and cycloheptyl radicals.

5. The regime or regimen as defined by claim 1, wherein the at least one compound of formula (I), R1 and R2, which may be identical or different are each a hydrogen atom or a C₁-C₇ alkyl radical or a C₃-C₇ cycloalkyl radical.

6. The regime or regimen as defined by claim 1, wherein said at least one compound of general formula (I) is selected from the group consisting of:
4-phenyl-1,3-dihydroimidazole-2-thione;
4-phenyl-1,3-dihydroimidazole-2-thione hydrochloride;
4-(4-methoxyphenyl)-1,3-dihydroimidazole-2-thione;
4-(3-bromophenyl)-1,3-dihydroimidazole-2-thione;
4-(4-bromophenyl)-1,3-dihydroimidazole-2-thione;
4-(2-fluorophenyl)-1,3-dihydroimidazole-2-thione;
4-(3-fluorophenyl)-1,3-dihydroimidazole-2-thione;
4-(4-fluorophenyl)-1,3-dihydroimidazole-2-thione;
4-(2-chlorophenyl)-1,3-dihydroimidazole-2-thione;
4-(3-chlorophenyl)-1,3-dihydroimidazole-2-thione;
4-(4-chlorophenyl)-1,3-dihydroimidazole-2-thione;
4-(4-butoxyphenyl)-1,3-dihydroimidazole-2-thione;
4-(4-methylphenyl)-1,3-dihydroimidazole-2-thione;
4-(4-trifluoromethylphenyl)-1,3-dihydroimidazole-2-thione;
4-(4-isopropylphenyl)-1,3-dihydroimidazole-2-thione;
4-(4-propylphenyl)-1,3-dihydroimidazole-2-thione;
4-(4-tert-butylphenyl)-1,3-dihydroimidazole-2-thione;
4-(3,4-dimethoxyphenyl)-1,3-dihydroimidazole-2-thione; and
4-(3-propyloxyphenyl)-1,3-dihydroimidazole-2-thione.

7. The regime or regimen as defined by claim 1, wherein said pigmentary disorders are selected from among melasma, chloasma, lentigines, senile lentigo, irregular hyperpigmentations related to photoaging, freckles, postinflammatory hyperpigmentations due to an abrasion and/or to a burn and/or to a scar and/or to a dermatosis and/or to a contact allergy, naevi, genetically determined hyperpigmentations, hyperpigmentations of metabolic or drug origin and melanomas.

8. The regime or regimen as defined by claim 1, for preventing and/or treating signs of skin aging.

9. The regime or regimen as defined by claim 1, for body or hair hygiene.
